(19) United States
(12) Patent Application Publication
(10) Pub. No.: US 2011/0237667 A1
(43) Pub. Date: Sep. 29, 2011

(54) Compositions Containing Diethanol Amine Esterquats

(75) Inventors: Matthias Loeffler, Idstein (DE); Franz-Xaver Scherl, Burgkirchen (DE); Tom Fricke, Liederbach (DE)

(73) Assignee: CLARIANT FINANCE (BVI) LIMITED, Tortola (VG)

(21) Appl. No.: 12/671,712

(22) PCT Filed: Jul. 31, 2008
(86) PCT No.: PCT/EP2008/006293

§ 371 Date: Jul. 29, 2010

(30) Foreign Application Priority Data
Aug. 6, 2007 (DE) 102007037018.2
Mar. 27, 2008 (DE) 102008015899.2
Jul. 31, 2008 (EP) PCT/EP2008/006293

(51) Int. Cl. A61K 31/225; C07C 229/24; A61K 8/92; A61Q 19/00; A61Q 5/00; A61P 17/00

(52) U.S. Cl. 514/547; 554/103

(57) ABSTRACT

The invention relates to compositions containing one or more compounds of the formula (1), wherein R¹CO and R²CO are linear or branched saturated acyl groups independent of each other, having 18 to 24 C atoms, and A⁻ is a counter-ion, and the total amount of C₁₈₋₂₃-alkyl COO groups is 40.0 wt.-% or more, based on all groups R¹COO— and R²COO—. The compositions are, for example, cosmetic, dermatological, or pharmaceutical compositions.

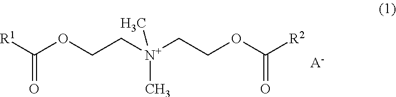

The invention relates to compositions comprising diethanolamine ester quats, wherein the two ester groups bear long acyl chains having 18 to 24 carbon atoms and preferably do not contain a carbon-carbon double bond, and the use thereof for skincare and haircare.

Skincare and haircare compositions comprising quaternary ammonium compounds which have at least one long-chain alkyl or alkenyl group, for example behenyltrimethylammonium chloride, stearyltrimethylammonium chloride, lauryltrimethylammonium chloride, cetyltrimethylammonium chloride, distearyldimethylammonium chloride, are known.

One disadvantage of this compound class is the poor biodegradability thereof. A significantly better biodegradability and simultaneously conditioning effects are exhibited by ester quats, whose ester groups derive from fatty acids.

EP 1 117 377 discloses cosmetic formulations which comprise mono-, di- and/or triester quats whose ester groups derive from C₈-C₁₈ coconut fatty acids.

EP 0 614 349 describes haircare compositions comprising quaternized fatty acid trialkanolamine ester salts with acyl groups, preferably derived from octadec-9-enoic acid with a content of trans-double bonds of 40 to 70% by weight.

WO 96/03970 discloses that diester quats whose ester groups derive from rapeseed oil and contain up to 3 double bonds can be used particularly advantageously in haircare compositions and feature improved conditioning effects and combability of the hair.

What is disadvantageous about the ester quats specified in the prior art is their inadequate stability, especially under the influence of light and oxygen. In addition, there is a demand for active ingredients for conditioning of the skin and of the hair, which exhibit good action even at low use amounts and have minor pollutant potential from an ecological viewpoint.

The objective was thus to remedy the disadvantages of the prior art or at least to reduce them and more particularly to develop active ingredients which have good formulability, exhibit improved conditioning effects and more particularly bring about better combability of wet and dry hair, reduce static charge, improve the feel, and impart shine and color protection to the hair. More particularly, there is a need for active ingredients which, in spite of good biodegradability, have outstanding conditioning properties and additionally good skin compatibility. The development of active ingredients should especially also include the possibility of providing the active ingredients in the form of compositions, which may already have been formulated.

It has now been found that, surprisingly, this objective is achieved by diethanolamine ester quats wherein the two ester groups bear long-chain saturated acyl radicals having 18 to 24 carbon atoms, and by specific compositions comprising such diethanolamine ester quats, and they especially exhibit improved effects with regard to combability, shine, color protection, antistatic properties and skinfeel. In addition, these diethanolamine ester quats are notable for significantly higher color stability compared to unsaturated ester quats. This stability has a positive effect on the storage of the diethanolamine ester quats, since color and odor do not change even in the course of prolonged storage at relatively high temperature. In a cosmetic formulation too, the diethanolamine ester quats have better stabilities in storage. In addition, these ester quats, when used in hair- and skincare compositions, bring about a relatively high viscosity and hence an improved stability of the cosmetic formulation. In spite of the good conditioning properties, the diethanolamine ester quats are found to be readily biodegradable.

The invention therefore provides compositions comprising one or more compounds of the formula (1)

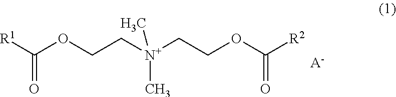

in which
R¹CO and R²CO are each independently linear or branched saturated acyl groups having 18 to 24 carbon atoms and
A⁻ is a counterion
and the total amount of C₁₈₋₂₃-alkyl-COO— groups, preferably the total amount of C₁₉₋₂₃-alkyl-COO— groups, based on all R¹COO— and R²COO— groups, is 40.0% by weight or more and preferably 42.0% by weight or more.

In the context of the present application, reference is made to compositions when a physical mixture of 2 or more chemical compounds is present. The inventive compositions may thus be mixtures of 2 or more compounds of the formula (1). However, they may also be compositions which contain only one or else more than one compound of the formula (1) and additionally one or more other compounds. These compositions may, for example, be compositions for end use, for example already formulated cosmetic, dermatological or pharmaceutical compositions.

The compounds of the formula (1) in which R¹CO and R²CO are each independently linear or branched saturated acyl groups having 18 to 24 carbon atoms can also be described such that R¹ and R² in formula (1) are each independently linear or branched alkyl groups having 17 to 23 carbon atoms.

The R¹ and R² radicals of the compounds of the formula (1) are preferably each independently linear alkyl groups having 17 to 23 carbon atoms.

In a preferred embodiment of the invention, the total amount of C₁₉-alkyl-COO—, C₂₁-alkyl-COO— and C₂₃-alkyl-COO— groups, based on all R¹COO— and R²COO— groups, is 40.0% by weight or more and preferably 42.0% by weight or more.

In a particularly preferred embodiment of the invention, the total amount of C₁₉-alkyl-COO— and C₂₁-alkyl-COO— groups, based on all R¹COO— and R²COO— groups, is 40.0% by weight or more and preferably 42.0% by weight or more.

In an especially preferred embodiment of the invention, the total amount of C₂₁-alkyl-COO— groups, based on all R¹COO— and R²COO— groups, is 40.0% by weight or more and preferably 42.0% by weight or more.

In an exceptionally preferred embodiment of the invention, a plurality of compounds of the formula (1) are present in the inventive compositions, and the amount of C₁₇-alkyl-COO— groups of the compounds of the formula (1), based on all R¹COO— and R²COO— groups, is from 1.0 to 45.0% by weight, the amount of C₁₉-alkyl-COO— groups of the compounds of the formula (1), based on all R¹COO— and R²COO— groups, is from 1.0 to 15.0% by weight, and the amount of C₂₁-alkyl-COO— groups of the compounds of the formula (1), based on all R¹COO— and R²COO— groups, is from 42.0 to 94.0% by weight.

In an extremely preferred embodiment of the invention, a plurality of compounds of the formula (1) are present in the inventive compositions, and the amount of C₁₇-alkyl-COO— groups of the compounds of the formula (1), based on all R¹COO— and R²COO— groups, is from 41.0 to 45.0% by weight, the amount of C₁₉-alkyl-COO— groups of the compounds of the formula (1), based on all R¹COO— and R²COO— groups, is from 9.0 to 13.0% by weight, and the amount of C₂₁-alkyl-COO— groups of the compounds of the formula (1), based on all R¹COO— and R²COO— groups, is from 42.0 to 46.0% by weight.

In a further extremely preferred embodiment of the invention, a plurality of compounds of the formula (1) are present in the inventive compositions, and the amount of C₁₇-alkyl-COO— groups of the compounds of the formula (1), based on all R¹COO— and R²COO— groups, is from 1.0 to 6.0% by weight, the amount of C₁₉-alkyl-COO— groups of the compounds of the formula (1), based on all R¹COO— and R²COO— groups, is from 12.0 to 15.0% by weight, and the amount of C₂₁-alkyl-COO— groups of the compounds of the formula (1), based on all R¹COO— and R²COO— groups, is from 79.0 to 84.0% by weight.

In a further extremely preferred embodiment of the invention, a plurality of compounds of the formula (1) are present in the inventive compositions, and the amount of C₁₇-alkyl-COO— groups of the compounds of the formula (1), based on all R¹COO— and R²COO— groups, is from 1.0 to 6.0% by weight, the amount of C₁₉-alkyl-COO— groups of the compounds of the formula (1), based on all R¹COO— and R²COO— groups, is from 1.0 to 6.0% by weight, and the amount of C₂₁-alkyl-COO— groups of the compounds of the formula (1), based on all R¹COO— and R²COO— groups, is from 90.0 to 94.0% by weight.

In a further preferred embodiment of the invention, the amount of C₂₃-alkyl-COO— groups of the compounds of the formula (1), based on all R¹COO— and R²COO— groups, in the inventive compositions is up to 3.0% by weight, preferably from 0.5 to 2.5% by weight and more preferably from 1.0 to 2.0% by weight.

In a further exceptionally preferred embodiment of the invention, a plurality of compounds of the formula (1) are present in the inventive compositions, and the amount of C₁₇-alkyl-COO— groups of the compounds of the formula (1), based on all R¹COO— and R²COO— groups, is from 1.0 to 5.0% by weight, the amount of C₁₉-alkyl-COO— groups of the compounds of the formula (1), based on all R¹COO— and R²COO— groups, is from 6.0 to 10.0% by weight, and the amount of C₂₁-alkyl-COO— groups of the compounds of the formula (1), based on all R¹COO— and R²COO— groups, is from 86.0 to 90.0% by weight. Among these inventive compositions, preference is given in turn to those in which the amount of C₂₃-alkyl-COO— groups of the compounds of the formula. (1), based on all R¹COO— and R²COO— groups, is from 1.0 to 5.0% by weight.

In a further preferred embodiment of the invention, the inventive compositions comprise one or more compounds of the formula (1) in which R¹ and R² are each linear or branched, preferably linear, alkyl groups having 21 carbon atoms.

Further preferred inventive compositions comprise one or more compounds of the formula (1) and one or more compounds of the formula (2)

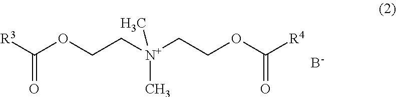

in which
R³CO and R⁴CO are each independently linear or branched saturated acyl groups having 12 to 24 carbon atoms, preferably having 18 to 24 carbon atoms, or linear or branched, mono- or polyunsaturated acyl groups having 12 to 24 carbon atoms, preferably having 18 to 24 carbon atoms, where at least one of the R³CO and R⁴CO groups must be a mono- or polyunsaturated acyl group, and
B⁻ is a counterion and
the amount of the compounds of the formula (2), based on the total amount of the compounds of the formulae (1) and (2), is less than 20.0% by weight, preferably less than 10.0% by weight, more preferably less than 5.0% by weight and especially preferably less than 1.0% by weight.

Exceptionally preferred inventive compositions are those in which no compound of the formula (2) is present.

In the compounds of the formulae (1) and (2), the counterions A⁻ or A⁻ and B⁻ are preferably each independently selected from chloride, bromide, methosulfate MeSO₄⁻ (where Me is methyl, CH₃), tosylate, phosphate, sulfate, hydrogensulfate, lactate and citrate, more preferably from chloride and methosulfate MeSO₄⁻. The counterions A⁻ or A⁻ and B⁻ are especially preferably chloride.

The quaternary ester pats of the formula (1) are notable for outstanding conditioning action.

In a preferred embodiment of the invention, the inventive compositions, based on the overall composition, contain from 0.1 to 10.0% by weight and preferably from 1.0 to 5.0% by weight of the one or more compounds of the formula (1). In this embodiment of the invention, the inventive compositions may, for example, be cosmetic, dermatological or pharmaceutical compositions.

Particularly advantageous performance properties are exhibited by inventive compositions comprising one or more unbranched or branched monoalcohols with an alkyl group having 8 to 22 carbon atoms, and preferably, based on the overall composition, from 0.1 to 70.0% by weight of one or more of these substances. Additionally preferred are therefore inventive compositions comprising one or more unbranched or branched monoalcohols with an alkyl group having 8 to 22 carbon atoms, and preferably, based on the overall composition, from 0.1 to 70.0% by weight of one or more of these substances.

Useful monoalcohols of this type are preferably lauryl alcohol, stearyl alcohol, cetyl alcohol, Guerbet alcohol and behenyl alcohol.

To improve the consistency of the composition, it may be advantageous to add one or more short-chain monoalcohols thereto.

In a further preferred embodiment of the invention, the composition therefore comprises one or more monoalcohols with an alkyl group having 1 to 7 carbon atoms, and preferably, based on the overall composition, from 0.1 to 70.0% by weight of one or more of these substances.

The monoalcohols used are preferably ethanol, propanol, isopropanol, butanol, isobutanol and t-butanol, more preferably isopropanol.

In a further preferred embodiment of the invention, the composition comprises one or more polyols having 3 to 12 carbon atoms, and preferably, based on the overall composition, from 0.1 to 70.0% by weight of one or more of these substances.

Preferred polyhydric alcohols, i.e. polyols, are pentanediol, hexanediol, heptanediol, octanediol, nonanediol, decanediol, undecanediol, dodecanediol, diglycerol, triglycerol, dipropylene glycol, tripropylene glycol, sorbitol, xylitol, mannitol and/or mixtures thereof. Particularly preferred polyhydric alcohols, i.e. polyols, are 1,5-pentanediol, 1,2-pentanediol, 1,6-hexanediol, 1,2-hexanediol, 1,7-heptanediol, 1,2-heptanediol, 1,8-octanediol, 1,2-octanediol, 1,9-nonanediol, 1,2-nonanediol, 1,10-decanediol, 1,2-decanediol, 1,11-undecanediol, 1,2-undecanediol, 1,12-dodecanediol, 1,2-dodecanediol, diglycerol, triglycerol, dipropylene glycol, tripropylene glycol, sorbitol, xylitol, mannitol and/or mixtures thereof.

In a further preferred embodiment of the invention, the inventive compositions comprise diethanol/methylamine and preferably, based on the overall composition, from 10 ppm to 1.0% by weight of diethanol/methylamine.

To regulate the consistency of the inventive compositions, small amounts of diethanol/methylamine and small amounts of one or more fatty acids of the formula R^{1a}COOH and R^{2a}COOH, in which R^{1a}CO and R^{2a}CO are each independently linear or branched acyl groups having 18 to 24 carbon atoms and preferably linear or branched saturated acyl groups having 18 to 24 carbon atoms, may be advantageous.

In a further preferred embodiment of the invention, the inventive compositions comprise one or more fatty acids of the formulae R^{1a}COOH and R^{2a}COOH, in which R^{1a}CO and R^{2a}CO are each independently linear or branched acyl groups having 18 to 24 carbon atoms, preferably linear or branched saturated acyl groups having 18 to 24 carbon atoms, and preferably, based on the overall composition, from 10 ppm to 1.0% by weight of one or more of these substances.

The conditioning action of the inventive composition can be enhanced by adding N-modified silicones.

In a further preferred embodiment of the invention, the inventive compositions comprise alkylmethicones, alkyldimethicones or one or more amodimethicones. Amodimethicones are siloxane polymers grafted with amino-functional groups. Amodimethicones are known, for example, under the trade names Dow Corning 2-8566 Amino Fluid (Dow Corning Corporation), Mirasil ADME (Rhodia), SilCare® Silicone SEA (Clariant) or Wacker-Belsil ADM 1100 (Wacker Chemie AG), have a molecular weight between 800 and 260 000 g/mol and correspond generally to the formula (3)

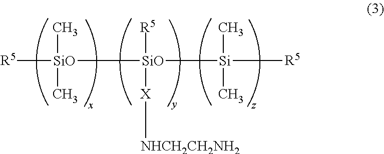

in which

### R⁵ is —OH or —CH₃,

X is a linear or branched C₁-C₆-alkylene group and
x, y and z are each independently numbers from 1 to 5500, preferably from 50 to 500.

In a particularly preferred embodiment of the invention, the inventive compositions comprise one or more of the abovementioned compounds of the formula (3), and preferably, based on the overall composition, 0.1 to 5.0% by weight of one or more compounds of the formula (3).

The compatibility with further ingredients, and the skinfeel and the antistatic effect of the composition, can be improved by adding one or more compounds of the formula (4)

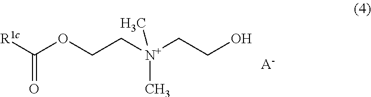

in which
R^{1c}CO is a linear or branched, preferably linear, additionally preferably saturated, acyl group having 18 to 24 carbon atoms, preferably 18 to 22 carbon atoms, and
A⁻ is a counterion.

In a further preferred embodiment of the invention, the inventive compositions comprise one or more compounds of the formula (4)

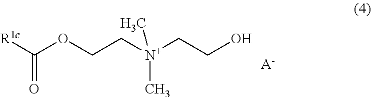

in which
R^{1c}CO is a linear or branched, preferably linear, additionally preferably saturated, acyl group having 18 to 24 carbon atoms, preferably 18 to 22 carbon atoms, and
A⁻ is a counterion,
and preferably, based on the overall composition, from 0.1 to 5.0% by weight of one or more compounds of the formula (4).

The counterion K of the formula (4) is preferably selected from chloride, bromide, methosulfate MeSO₄⁻, tosylate, phosphate, sulfate, hydrogensulfate, lactate and citrate, and is more preferably selected from chloride and methosulfate MeSO₄⁻.

The inventive compositions may, for example, be pellets, flakes, extrudates, pastes, compacts, powder, but also emulsions or dispersions.

In a further preferred embodiment of the invention, the inventive compositions are dispersions.

In a further preferred embodiment of the invention, the inventive compositions comprise one or more nonionic emulsifiers, and preferably, based on the overall composition, from 0.1 to 5.0% by weight of one or more nonionic emulsifiers.

Useful nonionic emulsifiers are preferably:

addition products of 0 to 30 mol of ethylene oxide and/or 0 to 5 mol of propylene oxide onto linear fatty alcohols having 8 to 22 carbon atoms, onto fatty acids having 12 to 22 carbon atoms, onto alkylphenols having 8 to 15 carbon atoms in the alkyl group and onto sorbitan or sorbitol esters; (C₁₂-C₁₈)-fatty acid mono- and diesters of addition products of 0 to 30 mol of ethylene oxide onto glycerol; glycerol mono- and diesters and sorbitan mono- and diesters of saturated and unsaturated fatty acids having 6 to 22 carbon atoms and optionally ethylene oxide addition products thereof; addition products of from 15 to 60 mol of ethylene oxide onto castor oil and/or hydrogenated castor oil; polyol and especially polyglycerol esters, for example polyglyceryl polyricinoleate and polyglyceryl poly-12-hydroxystearate. Ethoxylated fatty amines, fatty acid amides, fatty acid alkanolamides and mixtures of compounds of two or more of these substance classes are likewise preferably suitable.

Particular preference is given to using fatty alcohol ethoxylates selected from the group of ethoxylated stearyl alcohols, isotearyl alcohols, cetyl alcohols, isocetyl alcohols, oleyl alcohols, lauryl alcohols, isolauryl alcohols, cetylstearyl alcohols, especially polyethylene glycol(13) stearyl ether, polyethylene glycol(14) stearyl ether, polyethylene glycol(15) stearyl ether, polyethylene glycol(16) stearyl ether, polyethylene glycol(17) stearyl ether, polyethylene glycol(18) stearyl ether, polyethylene glycol(19) stearyl ether, polyethylene glycol(20) stearyl ether, polyethylene glycol(12) isostearyl ether, polyethylene glycol(13) isostearyl ether, polyethylene glycol(14) isostearyl ether, polyethylene glycol(15) isostearyl ether, polyethylene glycol(16) isostearyl ether, polyethylene glycol(17) isostearyl ether, polyethylene glycol(18) isostearyl ether, polyethylene glycol(19) isostearyl ether, polyethylene glycol(20) isostearyl ether, polyethylene glycol(13) cetyl ether, polyethylene glycol(14) cetyl ether, polyethylene glycol(15) cetyl ether, polyethylene glycol(16) cetyl ether, polyethylene glycol(17) cetyl ether, polyethylene glycol(18) cetyl ether, polyethylene glycol(19) cetyl ether, polyethylene glycol(20) cetyl ether, polyethylene glycol(13) isocetyl ether, polyethylene glycol(14) isocetyl ether, polyethylene glycol(15) isocetyl ether, polyethylene glycol(16) isocetyl ether, polyethylene glycol(17) isocetyl ether, polyethylene glycol(18) isocetyl ether, polyethylene glycol(19) isocetyl ether, polyethylene glycol(20) isocetyl ether, polyethylene glycol(12) oleyl ether, polyethylene glycol(13) oleyl ether, polyethylene glycol(14) oleyl ether, polyethylene glycol(15) oleyl ether, polyethylene glycol(12) lauryl ether, polyethylene glycol(12) isolauryl ether, polyethylene glycol(13) cetylstearyl ether, polyethylene glycol(14) cetylstearyl ether, polyethylene glycol(15) cetylstearyl, ether, polyethylene glycol(16) cetylstearyl ether, polyethylene glycol(17) cetylstearyl ether, polyethylene glycol(18) cetylstearyl ether, polyethylene glycol(19) cetylstearyl ether.

Equally preferred are fatty acid ethoxylates selected from the group of ethoxylated stearates, isostearates and oleates, especially polyethylene glycol(20) stearate, polyethylene glycol(21) stearate, polyethylene glycol(22) stearate, polyethylene glycol(23) stearate, polyethylene glycol(24) stearate, polyethylene glycol(25) stearate, polyethylene glycol(12) isostearate, polyethylene glycol(13) isostearate, polyethylene glycol(14) isostearate, polyethylene glycol(15) isostearate, polyethylene glycol(16) isostearate, polyethylene glycol(17) isostearate, polyethylene glycol(18) isostearate, polyethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate, polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, polyethylene glycol(20) oleate.

The ethoxylated alkyl ether carboxylic acid or salt thereof used may advantageously be sodium laureth-11 carboxylate.

The ethoxylated triglycerides used may advantageously be polyethylene glycol(60) evening primrose glycerides.

It is additionally advantageous to select the polyethylene glycol glycerol fatty acid esters from the group of polyethylene glycol(20) glyceryl laurate, polyethylene glycol(6) glyceryl caprate/caprinate, polyethylene glycol(20) glyceryl oleate, polyethylene glycol(20) glyceryl isostearate and polyethylene glycol(18) glyceryl oleate/cocoate.

Particularly suitable among the sorbitan esters are polyethylene glycol(20) sorbitan monolaurate, polyethylene glycol(20) sorbitan monostearate, polyethylene glycol(20) sorbitan monoisostearate, polyethylene glycol(20) sorbitan monopalmitate, polyethylene glycol(20) sorbitan monooleate.

Further useful substances are glyceryl monostearate, glyceryl monooleate, diglyceryl monostearate, glyceryl isostearate, polyglyceryl-3 oleate, polyglyceryl-3 diisostearate, polyglyceryl-4 isostearate, polyglyceryl-2 dipolyhydroxystearate, polyglyceryl-4 dipolyhydroxystearate, PEG-30 dipolyhydroxystearate, diisostearoyl polyglyceryl-3 diisostearate, glycol distearate and polyglyceryl-3 dipolyhydroxystearate, sorbitan monoisostearate, sorbitan stearate, sorbitan oleate, sucrose distearate, lecithin, PEG-7-hydrogenated castor oil, cetyl alcohol, stearyl alcohol, behenyl alcohol, isobehenyl alcohol and polyethylene glycol(2) stearyl ether (steareth-2), alkylmethicone copolyols and alkyldimethicone copolyols, especially cetyldimethicone copolyol, laurylmethiconecopolyol.

In a further preferred embodiment of the invention, the inventive compositions are cosmetic, dermatological or pharmaceutical compositions.

The inventive cosmetic, dermatological or pharmaceutical compositions are preferably care compositions for the skin, and hair treatment compositions.

Examples of preferred inventive cosmetic, dermatological or pharmaceutical compositions are 2-in-1 shower gels, shower creams, skincare compositions, day creams, night creams, care creams, nutrient creams, body lotions and ointments.

In a further preferred embodiment of the invention, the inventive compositions are oil-in-water emulsions, preferably oil-in-water emulsions for treatment or care of the skin.

In a particularly preferred embodiment of the invention, the inventive compositions are compositions for treatment or care of hair, for example shampoos, rinse-off hair conditioners, cream rinses, clear rinses, hair cures, hair colorants and hair tints, permanent wave compositions, hair gels, hair conditioners in aerosol, spray and fluid form.

The cosmetic, dermatological and pharmaceutical compositions may comprise, as further assistants and additives, all customary surfactants, oil bodies, cationic polymers, film formers, thickeners and gelating agents, superfatting agents, active antimicrobial and biogenic ingredients, humectants, stabilizers, preservatives, pearlizing agents, dyes and fragrances.

The surfactants used may be cationic, nonionic, amphoteric and/or zwitterionic surfactants.

Preferred cationic surfactants are quaternary ammonium salts, such as di(C₈-C₂₂)-alkyldimethylammonium chloride or bromide, preferably di(C₈-C₂₂)-alkyldimethylammonium chloride or bromide; (C₈-C₂₂)-alkyldimethylethyl-ammonium chloride or bromide; (C₈-C₂₂)-alkyltrimethylammonium chloride or bromide, preferably cetyltrimethylammonium chloride or bromide and (C₈-C₂₂)-alkyltrimethylammonium chloride or bromide; (C₁₀-C₂₄)-alkyldimethyl-benzylammonium chloride or bromide, preferably (C₁₂-C₁₈)-alkyldimethyl-benzylammonium chloride, (C₈-C₂₂)-alkyldimethylhydroxyethylammonium chloride, phosphate, sulfate, lactate, (C₈-C₂₂)-alkylamidopropyltrimethylammonium chloride, methosulfate, N,N-bis(2-C₈-C₂₂-alkanoyloxyethyl)dimethylammonium chloride, methosulfate, N,N-bis(2-C₈-C₂₂-alkanoyl-oxyethyl)hydroxyethylmethylammonium chloride, methosulfate.

The amount of the cationic surfactants is preferably from 0.1 to 10.0% by weight, more preferably from 0.5 to 7.0% by weight and especially preferably from 1.0 to 5.0% by weight, based on the finished compositions.

Preferred nonionic surfactants are fatty alcohol ethoxylates (alkylpolyethylene glycols); alkylphenol polyethylene glycols; fatty amine ethoxylates (alkylaminopolyethylene glycols); fatty acid ethoxylates (acyl polyethylene glycols); polypropylene glycol ethoxylates (Pluronics®); fatty acid alkanolamides, (fatty acid amide polyethylene glycols); sucrose esters; sorbitol esters and sorbitan esters and polyglycol ethers thereof, and also C₈-C₂₂-alkyl polyglucosides.

The amount of the nonionic surfactants in the inventive compositions (for example in the case of rinse-off products) is preferably in the range from 1.0 to 20.0% by weight, more preferably from 2.0 to 10.0% by weight and especially preferably from 3.0 to 7.0% by weight.

In addition, the inventive compositions may comprise amphoteric surfactants. These can be described as derivatives of long-chain secondary or tertiary amines which have an alkyl group with 8 to 18 carbon atoms and in which a further group is substituted by an anionic group which imparts the solubility in water, thus, for example, by a carboxyl, sulfate or sulfonate group. Preferred amphoteric surfactants are N—(C₁₂-C₁₈)-alkyl-β-aminopropionates and N—(C₁₂-C₁₈)-alkyl-β-iminodipropionates as alkali metal and mono-, di- and trialkylammonium salts; suitable further surfactants are also amine oxides. These are oxides of tertiary amines with a long-chain group having 8 to 18 carbon atoms and two mostly short-chain alkyl groups having 1 to 4 carbon atoms. Preference is given here, for example, to the C₁₀- to C₁₈-alkyldimethylamine oxides, fatty acid amidoalkyldimethylamine oxide.

A further preferred group of surfactants is betaine surfactants, also known as zwitterionic surfactants. These contain, in the same molecule, a cationic group, especially an ammonium group, and an anionic group, which may be a carboxylate group, sulfate group or sulfonate group. Suitable betaines are preferably alkylbetaines such as cocobetaine or fatty acid alkylamidopropylbetaines, for example cocoacylamidopropyldimethylbetaine or the C₁₂- to C₁₈-dimethylaminohexanoates and/or the C₁₀- to C₁₈-acylamidopropanedimethylbetaines.

The amount of the amphoteric surfactants and/or betaine surfactants is preferably from 0.5 to 20.0% by weight and more preferably from 1.0 to 10.0% by weight.

Preferred surfactants are cocoamidopropylbetaine, alkylbetaines such as cocobetaine, sodium cocoyl glutamate and lauroamphoacetate.

In a preferred embodiment, the inventive compositions additionally also comprise, as foam-boosting agents, cosurfactants from the group of alkylbetaines, alkylamidobetaines, aminopropionates, aminoglycinates, imidazolinium betaines and sulfobetaines, amine oxides, fatty acid alkanolamides and polyhydroxyamides.

The oil bodies may advantageously be selected from the group of triglycerides, natural and synthetic fatty substances, preferably esters of fatty acids with alcohols of low carbon number, for example with isopropanol, propylene glycol or glycerol, or esters of fatty alcohols with alkanoic acids of low carbon number or with fatty acids or from the group of alkyl benzoates, and also natural or synthetic hydrocarbon oils.

Useful substances include triglycerides of linear or branched, saturated or unsaturated, optionally hydroxylated, C₈-C₃₀-fatty acids, in particular vegetable oils, such as sunflower oil, corn oil, soybean oil, rice oil, jojoba oil, babussu oil, pumpkin oil, grapeseed oil, sesame oil, walnut oil, apricot oil, orange oil, wheatgerm oil, peach kernel oil, macadamia oil, avocado oil, sweet almond oil, lady's smock oil, castor oil, olive oil, peanut oil, rapeseed oil and coconut oil, and also synthetic triglyceride oils, e.g. the commercial product Myritol® 318. Hydrogenated triglycerides are also preferred in accordance with the invention. Oils of animal origin, for example beef tallow, perhydrosqualene, lanolin, can also be used.

A further class of oil bodies preferred in accordance with the invention is that of the benzoic esters of linear or branched C₈₋₂₂-alkanols, e.g. the commercial products Finsolv® SB (isostearyl benzoate), Finsolv® TN(C₁₂-C₁₅-alkyl benzoate) and Finsolv® EB (ethylhexyl benzoate).

A further class of oil bodies preferred in accordance with the invention is that of the dialkyl ethers having a total of 12 to 36 carbon atoms, especially having 12 to 24 carbon atoms, for example di-n-octyl ether (Cetiol® OE), di-n-nonyl ether, di-n-decyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether, di-3-ethyldecyl ether, tert-butyl n-octyl ether, isopentyl n-octyl ether and 2-methylpentyl n-octyl ether, and di-tert-butyl ether and diisopentyl ether.

Likewise useful are branched saturated or unsaturated fatty alcohols having 6-30 carbon atoms, e.g, isostearyl alcohol, and Guerbet alcohols.

A further class of oil bodies preferred in accordance with the invention is that of alkyl hydroxycarboxylates. Preferred alkyl hydroxycarboxylates are full esters of glycolic acid, lactic acid, malic acid, tartaric acid or citric acid. Further esters of hydroxycarboxylic acids which are suitable in principle are esters of p-hydroxypropionic acid, of tartronic acid, of D-gluconic acid, sugar acid, mucic acid or glucuronic acid. Suitable alcohol components of these esters are primary, linear or branched aliphatic alcohols having 8 to 22 carbon atoms. The esters of C₁₂-C₁₅-fatty alcohols are particularly preferred. Esters of this type are commercially available, e.g. under the trade name Cosmacol® from EniChem, Augusta Industriale.

A further class of oil bodies preferred in accordance with the invention is that of dicarboxylic esters of linear or branched C₂-C₁₀-alkanols, such as di-n-butyl adipate (Cetiol® B), di(2-ethylhexyl) adipate and di(2-ethylhexyl) succinate, and also diol esters, such as ethylene glycol dioleate, ethylene glycol diisotridecanoate, propylene glycol di(2-ethylhexanoate), propylene glycol diisostearate, propylene glycol dipelargonate, butanediol diisostearate and neopentyl glycol dicaprylate, and also diisotridecyl azelate.

Likewise preferred oil bodies are symmetrical, asymmetrical or cyclic esters of carbonic acid with fatty alcohols, glycerol carbonate or dicaprylyl carbonate (Cetiol® CC).

A further class of oil bodies preferred in accordance with the invention is that of the esters of dimers of unsaturated C₁₂-C₂₂-fatty acids (dimer fatty acids) with monovalent linear, branched or cyclic C₂-C₁₈-alkanols or with polyvalent linear or branched C₂-C₆-alkanols.

A further class of oil bodies preferred in accordance with the invention is that of hydrocarbon oils, for example those with linear or branched, saturated or unsaturated C₇-C₄₀-carbon chains, for example Vaseline, dodecane, isododecane, cholesterol, lanolin, synthetic hydrocarbons such as polyolefins, in particular polyisobutene, hydrogenated polyisobutene, polydecane, and hexadecane, isohexadecane, paraffin oils, isoparaffin oils, e.g. the commercial products of the Permethyl® series, squalane, squalene, and alicyclic hydrocarbons, e.g. the commercial product 1,3-di(2-ethylhexyl)cyclohexane (Cetiol® S), ozokerite, and ceresine.

Available silicone oils and silicone waxes are preferably dimethylpolysiloxanes and cyclomethicones, polydialkylsiloxanes R₃SiO(R₂SiO)ₓSiR₃ where R is methyl or ethyl, more preferably methyl, and x is a number from 2 to 500, for example the dimethicones available under the trade names VICASIL (General Electric Company), DOW CORNING 200, DOW CORNING 225, DOW CORNING 200 (Dow Corning Corporation), and also the dimethicones available under SilCare® Silicone 41M65, SilCare® Silicone 41M70, SilCare® Silicone 41M80 (Clariant), stearyldimethylpolysiloxane, C₂₀-C₂₄-alkyldimethylpolysiloxane, C₂₄-C₂₈-alkyldimethylpolysiloxane, but also the methicones available as SilCare® Silicone 41M40, SilCare® Silicone 41M50 (Clariant), and also trimethylsiloxysilicates [(CH₂)₃SiO)_{1/2}]ₓ[SiO₂]_{y}, where x is a number from 1 to 500 and y is a number from 1 to 500, dimethiconols R₃SiO[R₂SiO]ₓSiR₂OH and HOR₂SiO[R₂SiO]ₓSiR₂OH, where R is methyl or ethyl and x is a number up to 500, polyalkylarylsiloxanes, for example the polymethylphenylsiloxanes available under the trade names SF 1075 METHYLPHENYL FLUID (General Electric Company) and 556 COSMETIC GRADE PHENYL TRIMETHICONE FLUID (Dow Corning Corporation), polydiarylsiloxanes, silicone resins, cyclic silicones and amino-, fatty acid-, alcohol-, polyether-, epoxy-, fluorine- and/or alkyl-modified silicone compounds, and also polyether siloxane copolymers.

Suitable cationic polymers are those known by the INCI name “Polyquaternium”, in particular Polyquaternium-31, Polyquaternium-16, Polyquaternium-24, Polyquaternium-7, Polyquaternium-22, Polyquaternium-39, Polyquaternium-28, Polyquaternium-2, Polyquaternium-10, Polyquaternium-11, and Polyquaternium 37&mineral oil&PPG trideceth (Salcare SC95), PVP-dimethylaminoethyl methacrylate copolymer, guar hydroxypropyltriammonium chloride, and calcium alginate and ammonium alginate. It is additionally possible to use cationic cellulose derivatives; cationic starch; copolymers of diallylammonium salts and acrylamides; quaternized vinylpyrrolidone/vinylimidazole polymers; condensation products of polyglycols and amines; quaternized collagen polypeptides; quaternized wheat polypeptides; polyethyleneimines; cationic silicone polymers, for example amidomethicones; copolymers of adipic acid and dimethylaminohydroxypropyldiethylenetriamine; polyaminopolyamide and cationic chitin derivatives, for example chitosan.

The inventive compositions may comprise one or more of the above-mentioned cationic polymers in amounts of 0.1 to 5.0% by weight, preferably of 0.2 to 3.0% by weight and more preferably of 0.5 to 2.0% by weight, based on the finished compositions.

In addition, the inventive compositions may comprise film formers which, depending on the intended use, are selected from salts of phenylbenzimidazolesulfonic acid, water-soluble polyurethanes, for example C₁₀-polycarbamyl polyglyceryl ester, polyvinyl alcohol, polyvinylpyrrolidone copolymers such as PVP/hexadecene or PVP/eicosene copolymer, for example vinylpyrrolidone/vinyl acetate copolymer, water-soluble acrylic acid polymers/copolymers and esters or salts thereof, for example partial ester copolymers of acrylic acid/methacrylic acid and polyethylene glycol ethers of fatty alcohols, such as Acrylate/Steareth-20-Methacrylate Copolymer, water-soluble cellulose, for example hydroxymethylcellulose, hydroxyethylcellulose, hydroxypropylcellulose, water-soluble quaterniums, polyquaterniums, carboxyvinyl polymers, such as carbomers and salts thereof, polysaccharides, for example polydextrose and glucan, vinyl acetate/crotonate, for example available under the trade name Aristoflex® A 60 (Clariant), and polymeric amine oxides, for example representatives obtainable under the trade names Diaformer Z-711, 712, 731, 751.

The inventive compositions may comprise one or more film formers in amounts of 0.1 to 10.0% by weight, preferably of 0.2 to 5.0% by weight and more preferably of 0.5 to 3.0% by weight, based on the finished compositions.

The desired viscosity of the compositions can be established by adding thickeners and gelating agents. Useful substances are preferably cellulose ethers and other cellulose derivatives (e.g. carboxymethylcellulose, hydroxyethylcellulose), gelatin, starch and starch derivatives, sodium alginates, fatty acid polyethylene glycol esters, agar agar, tragacanth or dextrin derivatives, in particular dextrin esters. Additionally suitable are metal salts of fatty acids, preferably having 12 to 22 carbon atoms, for example sodium stearate, sodium palmitate, sodium laurate, sodium arachidate, sodium behenate, potassium stearate, potassium palmitate, sodium myristate, aluminum monostearate, hydroxyl fatty acids, for example 12-hydroxystearic acid, 16-hydroxyhexadecanoyl acid; fatty acid amides; fatty acid alkanolamides; dibenzalsorbitol and alcohol-soluble polyamides and polyacrylamides or mixtures of such. It is also possible to use crosslinked and uncrosslinked polyacrylates such as carbomers, sodium polyacrylates or polymers containing sulfonic acid, such as ammonium acryloyldimethyltaurate/VP copolymer.

The inventive compositions preferably contain from 0.01 to 20.0% by weight, more preferably from 0.1 to 10.0% by weight, especially preferably from 0.2 to 3.0% by weight and most preferably from 0.4 to 2.0% by weight of thickeners and/or gelating agents.

The superfatting agents used may preferably be lanolin and lecithin, nonethoxylated and polyethoxylated or acylated lanolin derivatives and lecithin derivatives, polyol fatty acid esters, mono-, di- and triglycerides and/or fatty acid alkanolamides, where the latter simultaneously serve as foam stabilizers, which are preferably used in amounts of 0.01 to 10.0% by weight, more preferably of 0.1 to 5.0% by weight and especially preferably of 0.5 to 3.0% by weight.

The active antimicrobial ingredients used are cetyltrimethylammonium chloride, cetylpyridinium chloride, benzethonium chloride, diisobutylethoxyethyldimethylbenzylammonium chloride, sodium N-laurylsarcosinate, sodium N-palmethylsarcosinate, lauroylsarcosine, N-myristoylglycine, potassium N-laurylsarcosine, trimethylammonium chloride, sodium aluminum chlorohydroxylactate, triethyl citrate, tricetylmethylammonium chloride, 2,4,4′-trichloro-2′-hydroxydiphenyl ether (triclosan), phenoxyethanol, 1,5-pentanediol, 1,6-hexanediol, 3,4,4′-trichlorocarbanilide (triclocarban), diaminoalkylamide, for example L-lysine hexadecylamide, citrate heavy metal salts, salicylates, piroctoses, in particular zinc salts, pyrithiones and heavy metal salts thereof, in particular zinc pyrithione, zinc phenol sulfate, farnesol, ketoconazole, oxiconazole, bifonazole, butoconazole, cloconazole, clotrimazole, econazole, enilconazole, fenticonazole, isoconazole, miconazole, sulconazole, tioconazole, fluconazole, itraconazole, terconazole, naftifine and terbinafine, selenium disulfide and Octopirox®, iodopropynyl butylcarbamate, methylchloroisothiazolinone, methylisothiazolinone, methyldibromoglutaronitrile, AgCl, chloroxylenol, sodium salt of diethylhexyl sulfosuccinate, sodium benzoate, and phenoxyethanol, benzyl alcohol, phenoxyisopropanol, parabens, preferably butyl, ethyl, methyl and propyl paraben, and sodium salts thereof, pentanediol, 1,2-octanediol, 2-bromo-2-nitropropane-1,3-diol, ethylhexylglycerol, benzyl alcohol, sorbic acid, benzoic acid, lactic acid, imidazolidinylurea, diazolidinylurea, dimethyloldimethylhydantoin (DMDMH), sodium salt of hydroxymethylglycinate, hydroxyethylglycine of sorbic acid and combinations of these active substances.

The inventive compositions comprise the active antimicrobial ingredients preferably in amounts of 0.001 to 5.0% by weight, more preferably of 0.01 to 3.0% by weight and especially preferably of 0.1 to 2.0% by weight, based on the finished compositions.

The inventive compositions may further comprise active biogenic ingredients selected from plant extracts, for example aloe vera, and also local anesthetics, antibiotics, antiphlogistics, antiallergics, corticosteroids, sebostatics, Bisabolol®, Allantoin®, Phytantriol®, proteins, vitamins selected from niacin, biotin, vitamin B2, vitamin B3, vitamin B6, vitamin B3 derivatives (salts, acids, esters, amides, alcohols), vitamin C and vitamin C derivatives (salts, acids, esters, amides, alcohols), preferably as sodium salt of the monophosphoric acid ester of ascorbic acid or as magnesium salt of the phosphoric acid ester of ascorbic acid, tocopherol and tocopherol acetate, and also vitamin E and/or derivatives thereof.

The inventive compositions may comprise active biogenic ingredients preferably in amounts of 0.001 to 5.0% by weight, more preferably 0.01 to 3.0% by weight and especially preferably 0.1 to 2.0% by weight, based on the finished compositions.

The inventive compositions may comprise astringents, preferably magnesium oxide, aluminum oxide, titanium dioxide, zirconium dioxide and zinc oxide, oxide hydrates, preferably aluminum oxide hydrate (boehmite) and hydroxides, preferably of calcium, magnesium, aluminum, titanium, zirconium or zinc, and also aluminum chlorohydrates, preferably in amounts of from 0 to 50.0% by weight, more preferably in amounts of from 0.01 to 10.0% by weight and especially preferably in amounts of from 0.1 to 10.0% by weight. Allantoin and bisabolol are preferred as deodorizing substances. These are preferably used in amounts of from 0.0001 to 10.0% by weight.

Examples of humectant substances available are isopropyl palmitate, glycerol and/or sorbitol.

The stabilizers used may be metal salts of fatty acids, for example magnesium stearate, aluminum stearate and/or zinc stearate, preferably in amounts of 0.1 to 10.0% by weight, preferably of 0.5 to 8.0% by weight and more preferably of 1.0 to 5.0% by weight, based on the finished compositions.

Suitable preservatives are, for example, phenoxyethanol, formaldehyde solution, parabens, pentanediol and sorbic acid.

Preferentially suitable pearlizing components are fatty acid monoalkanolamides, fatty acid dialkanolamides, monoesters or diesters of alkylene glycols, in particular ethylene glycol and/or propylene glycol or oligomers thereof, with higher fatty acids, for example palmitic acid, stearic acid and behenic acid, monoesters or polyesters of glycerol with carboxylic acids, fatty acids and metal salts thereof, ketosulfones or mixtures of the specified compounds. Particular preference is given to ethylene glycol distearates and/or polyethylene glycol distearates having an average of 3 glycol units.

If the inventive compositions comprise pearlizing compounds, they are preferably present in the inventive compositions in an amount of 0.1 to 15.0% by weight and more preferably in an amount of from 1.0 to 10.0% by weight.

The fragrance and/or perfume oils used may be individual odorant compounds, e.g. the synthetic products of the ester, ether, aldehyde, ketone, alcohol and hydrocarbon types. Odorant compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allyl cyclohexyl propionate, styrallyl propionate and benzyl salicylate. The ethers include, for example, benzyl ethyl ethers, the aldehydes include, for example, the linear alkanals having 8 to 18 carbon atoms, citral, citronellal, citronellyloxyacetaldehyde, cyclamenaldehyde, hydroxycitronellal, lilial and bourgeonal, the ketones include, for example, the ionones, alpha-isomethylionone and methyl cedryl ketone, the alcohols include anethol, citronellol, eugenol, geraniol, linalool, phenylethyl alcohol and terpineol, and the hydrocarbons include primarily the terpenes and balsams. Preference is given to using mixtures of different odorants which together produce a pleasing scent note.

Perfume oils may also include natural odorant mixtures, as obtainable from vegetable or animal sources, e.g. pine oil, citrus oil, jasmine oil, lily oil, rose oil or ylang-ylang oil. Essential oils of relatively low volatility, which in most cases are used as aromatic components, are also suitable as perfume oils, e.g. sage oil, chamomile oil, clove oil, melissa oil, mint oil, cinnamon leaf oil, linden blossom oil, juniperberry oil, vetiver oil, olibanum oil, galbanum oil and ladanum oil.

The inventive compositions are preferably adjusted to a pH in the range from 2 to 12, preferably in the range from 3 to 9.

The acids or alkalis used for adjusting the pH are preferably mineral acids, especially HCl, inorganic bases, especially NaOH or KOH, or organic acids, especially citric acid.

The total proportion of assistants and additives in the compositions or in the inventive compositions is preferably from 1.0 to 50.0% by weight and more preferably from 5.0 to 40.0% by weight.

In specific embodiments, the inventive compositions can also be converted to finished forms without any problem.

The inventive compositions may thus, for example, also be materials convertible to finished forms, or else—as already mentioned—pellets or flakes produced from these materials.

In a further preferred embodiment of the invention, the inventive compositions are materials which can be converted to finished forms, preferably to pellets or to flakes, which have low melting points not exceeding 100° C.

These materials which can be converted to finished forms can, for example, also be used advantageously as a base for cream rinses.

The inventive materials which can be converted to finished forms are preferably processed to give pellets or flakes. These inventive pellets or flakes preferably likewise have low melting points not exceeding 100° C.

The inventive materials which can be converted to finished forms and the inventive pellets or flakes, preferably the inventive pellets or flakes, contain preferably from 45.0 to 70.0% by weight of one or more compounds of the formula (1) and from 30.0 to 55.0% by weight of one or more monoalcohols with an unbranched or branched alkyl group having 8 to 22 carbon atoms and more preferably from 47.0 to 60.0% by weight of one or more compounds of the formula (1) and from 40.0 to 53.0% by weight of one or more monoalcohols with an unbranched or branched alkyl group having 8 to 22 carbon atoms. Among the monoalcohols, cetyl alcohol, stearyl alcohol and combinations thereof are preferred.

In a particularly preferred embodiment of the invention, the inventive materials which can be converted to finished forms and the inventive pellets or flakes, preferably the inventive pellets or flakes, are free of monoalcohols having 1 to 7 carbon atoms and polyols having 3 to 12 carbon atoms. Among these inventive materials which can be converted to finished forms and the inventive pellets or flakes, preferably the inventive pellets or flakes, preference is given in turn to those which consist of the compounds of the formula (1) and the monoalcohols with an alkyl group having 8 to 22 carbon atoms.

In a further particularly preferred embodiment of the invention, the inventive materials which can be converted to finished forms and the inventive pellets or flakes, preferably the inventive pellets or flakes, contain one or more compounds selected from monoalcohols having 1 to 7 carbon atoms and polyols having 3 to 12 carbon atoms in an amount of up to 10.0% by weight and preferably of 1.0 to 6.0% by weight. Preferred additives are isopropyl alcohol and dipropylene glycol. Especially the short-chain monoalcohols having 1 to 7 carbon atoms can contribute to improving the convertibility to finished forms.

The inventive pellets or flakes can advantageously be used to produce cosmetic, dermatological or pharmaceutical compositions or formulations.

The inventive compositions, especially the inventive cosmetic, dermatological or pharmaceutical compositions, are advantageously suitable for treatment or care of the skin.

The present invention therefore further provides for the use of the inventive compositions, especially of the inventive cosmetic, dermatological or pharmaceutical compositions, for treatment or care of the skin.

The inventive compositions, especially the inventive cosmetic, dermatological or pharmaceutical compositions, are additionally advantageously suitable for treatment or care of hair.

The present invention therefore further provides for the use of the inventive compositions, especially of the inventive cosmetic, dermatological or pharmaceutical compositions, for treatment or care of hair.

Preference is given to the use of the inventive cosmetic, dermatological or pharmaceutical compositions for conditioning hair.

The examples which follow serve to illustrate the invention, but without restricting it thereto.

The percentages reported in the examples are percent by weight (% by weight), unless explicitly stated otherwise.

### PREPARATION EXAMPLES AND APPLICATION EXAMPLES

### I. Preparation of Compounds of the Formula (1)

The diethanolamine ester quats of the formula (1) can be obtained by various relevant methods in preparative organic chemistry. Reviews on this subject have been published, for example, by R. Puchta et al. in Tens. Surf. Det., 30, 186 (1993), M. Brock in Tens. Surf. Det. 30, 394 (1993), R. Lagerman et al. in J. Am. Oil. Chem. Soc., 71, 97 (1994).

Preparation example I.1. which follows thus represents only one of several possible preparations. The alkylation can also be performed, alternatively to methyl chloride, with dimethyl sulfate, alkyl p-toluenesulfonate, etc.

### I.1. Preparation of the Compound of the Formula (1) where R¹, R²=n-C₂₁H₄₃ and A⁻=Cl⁻

1020.0 g of behenic acid n-C₂₁H₄₃—COOH (3.0 mol), 178.8 g of methyldiethanolamine (1.5 mol) are initially charged in the presence of acidic catalyst in a 2 liter esterification apparatus with water separator and condenser. Under a nitrogen blanket, the mixture is stirred at 160-190° C. for approx. 10 hours.

To quaternize the methyldiethanolamine dibehenate, 307.2 g of methyldiethanolamine dibehenate (0.4 mol) are taken up in isopropanol and initially charged in a 1 liter glass autoclave. The autoclave is heated to approx. 70° C., and 20.2 g of methyl chloride (0.4 mol) are injected in portions until the pressure is constant. Subsequently, the pressure is released, the product is transferred to a flask and isopropanol is distilled off on a rotary evaporator.

### I.2. Preparation of Further Compounds of the Formula (1)

Fatty acids used, which were converted to diethanolamine ester quats analogously to preparation example 1.1.:
(a) octadecanoic acid (stearic acid) C₁₈H₃₆O₂
(b) eicosanoic acid (arachic acid) C₂₀H₄₀O₂
(c) docosanoic acid (behenic acid) C₂₂H₄₄O₂ (used in Example 1.1.)
(d) tetracosanoic acid (lignoceric acid) C₂₄H₄₈O₂
(e) C₂₀₋₂₂ fatty acid (e.g. Edenor® C₂₂ 85R from Cognis, Prifac® 2989 from Uniqema) with C₁₈ not more than 5%, C₂₀ not more than 12%, C₂₂ not less than 85%, iodine number not more than 3
(f) C₁₈₋₂₂ fatty acid with C₁₈ 43%, C₂₀ 11%, O₂₂ 45%, C₂₄ 1%, iodine number not more than 3
(g) C₂₀₋₂₂ fatty acid with C₁₈ 3%, O₂₀ 13%, O₂₂ 82%, C₂₄ 2%, iodine number not more than 3
(h) C₂₂ fatty acid with C₁₈ 2%, C₂₀ 5%, C₂₂ 91%, C₂₄ 2%, iodine number not more than 3
(i) C₂₀₋₂₂ fatty acid with C₁₈ 2%, C₂₀ 8%, O₂₂ 88%, O₂₄ 2%, iodine number not more than 3

This gives rise to compounds of the formula (1) or compositions comprising compounds of the formula (1)

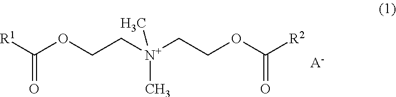

in which
A⁻ is Cl⁻ and R¹CO and R²CO is derived from
(1a) octadecanoic acid: Distearoylethyl Dimonium Chloride
(1b) eicosanoic acid: Dieicosoylethyl Dimonium Chloride
(1c) docosanoic acid: Didocosoylethyl Dimonium Chloride
(1d) tetracosanoic acid: Ditetracosoylethyl Dimonium Chloride
(1e) C₂₀₋₂₂ fatty acid: Dibehenoylethyl Dimonium Chloride
(1f) C₁₈₋₂₂ fatty acid: Stearoylethyl Behenoylethyl Dimonium Chloride
(1 g) C₂₀₋₂₂ fatty acid: Dibehenoylethyl Dimonium Chloride
(1 h) C₂₂ fatty acid: Dibehenoylethyl Dimonium Chloride
(1i) C₂₀₋₂₂ fatty acid: Dibehenoylethyl Dimonium Chloride

In the case of use of the fatty acids (e), (f), (g), (h) and (i), in each case only the name of the main product of the formula (1) is specified.

To prepare the compounds of the formula (1) or the compositions comprising compounds of the formula (1), it is also possible to use mixtures of the fatty acids (a) to (i).

### II. Production of Materials which can be Converted to Finished Forms, Pellets or Flakes Comprising Compounds of the Formula (1)

The quaternization can be effected, instead of in isopropanol, also in one or more monoalcohols with an unbranched or branched alkyl group having 8 to 22 carbon atoms, preferably in cetyl and/or stearyl alcohol. The compounds of the formula (1) resulting from the reaction, without or with compounds of the formula (2), can be melted below 100° C. without any problem in a weight ratio of 1:1 with any desired mixture of cetyl alcohol and stearyl alcohol, and be pelletized or converted to flake form. The weight ratio of cetyl alcohol to stearyl alcohol is preferably from 1:3 to 3:1.

Preparation example II.1. which follows gives one possible preparation of the inventive materials which can be converted to finished forms.

### II.1. Production of Materials which can be Converted to Finished Forms, Comprising a Compound of the Formula (1) where R¹, R²=n-C₂₁H₄₃ and A⁻=Cl⁻

To produce the materials which can be converted to finished forms, the quaternization of the methyldiethanolamine dibehenate is performed according to Example I.1. directly in mixtures of cetyl and stearyl alcohol (see Table 1).

TABLE 1
Mixtures of cetyl and stearyl alcohol in % by weight

| Mixture No. | Cetyl alcohol | Stearyl alcohol |
|---|---|---|
| 1 | 100% | — |
| 2 | 70% | 30% |
| 3 | 50% | 50% |
| 4 | 30% | 70% |
| 5 | — | 100% |

153.6 g of methyldiethanolamine dibehenate according to Example I.1. (0.2 mol) are initially charged in 163.7 g of mixtures 1-5 from Table 1 in a 1 liter glass autoclave. The autoclave is heated to approx. 85° C., and 10.1 g of methyl chloride (0.2 mol) are injected in portions until the pressure is constant. Subsequently, the pressure is released.

### II.2. Preparation of Materials which can be Converted to Finished Forms, Comprising Further Compounds of the Formula (1)

Analogously to Example II.1., materials which can be converted to finished forms are produced, except that, instead of 0.2 mol of methyldiethanolamine dibehenate according to Example I.1., 0.2 mol of methyldiethanolamine difatty acid ester which is the parent compound of the compounds (1b) and (1d) to (1i) specified in Example I.2. is used. For each of these methyldiethanolamine difatty acid esters, the preparation of the materials which can be converted to finished forms is performed with mixtures 1-5 from Table 1.

### II.3. Production of Pellets Comprising Compounds of the Formula (1)

To convert the resulting materials which can be converted to finished forms from Examples II.1. and II.2. to pellets, the molten material which can be converted to finished forms is in each case applied dropwise to a cooled continuous belt, such that it solidifies in the form of pellets.

### II.4. Production of Flakes Comprising Compounds of the Formula (1)

To convert the resulting materials which can be converted to finished forms from Examples II.1. and II.2. to flakes, the molten material which can be converted to finished forms is in each case applied to a cooled continuous belt so as to form a film of the particular molten material which can be converted to finished forms. The film is left to solidify and comminuted with a blade to flakes.

### III. Formulation Examples with Compounds of the Formula (1)

### Example 1

### Hair Rinse

| | | |
|---|---|---|
| A | Hostacerin ® T-3 (Clariant) | 1.50% |
| | Ceteareth-3 | 3.00% |
| | Cetyl Alcohol | |
| B | Water | ad 100.00% |
| C | Genamin ® KDMP (Clariant) | 2.50% |
| | Behentrimonium Chloride | |
| | Diethanolamine ester quat (according to Ex. 1e) | 3.00% |
| D | Fragrance | 0.30% |
| | Dye solution | q.s. |
| | Preservative | q.s. |
| E | Citric acid (10% in water) | q.s. |

### Preparation Method:

### I Melt A at 75° C.

II Dissolve C in B with stirring at approx. 75° C.
III Add II to I with stirring and cool while stirring.

### IV Add D to III at 35° C.

V Adjust to pH 4.0 with E.

### Example 2

### Hair Rinse with Vitamins and Uv Protection, Leave on

| | | |
|---|---|---|
| A | Propylene glycol | 0.70% |
| B | Octopirox ® (Clariant) | 0.05% |
| | Piroctone Olamine | |
| C | Carbopol ® 980 | 0.60% |
| | Carbomer | |
| D | Diethanolamine ester quat (according to Ex. 1e) | 0.20% |
| | Genamin ® CTAC (Clariant) | 0.50% |
| | Cetrimonium Chloride | |
| | Wheat starch | 0.50% |
| | Belsil DMC 6032 | 0.80% |
| | Dimethicone Copolyol Acetate | |
| | Dow Corning ® 190 | 0.50% |
| | Dimethicone Copolyol | |
| E | Water | ad 100.00% |
| | Panthenol | 0.20% |
| | Celquat ® L 200 | 0.10% |
| | Polyquaternium-4 | |
| | Uvinul MS 40 | 0.05% |
| | Benzophenone-4 | |
| | Nicotinamide | 0.30% |
| | Niacinamide | |
| | Tocopheryl Acetate | 0.10% |
| | Sodium hydroxide solution (50% in water) | 0.80% |
| F | Cosmedia ® Guar C 261 | 0.20% |
| | Guar Hydroxypropyltrimonium Chloride | |
| G | Fragrance | 0.30% |
| | Preservative | q.s. |

### Preparation Method:

### I Dissolve B in A.

II Melt D at approx. 80° C.
III Stir F into E with vigorous stirring,

### IV Add I to III.

### V Heat IV to 80° C.

VI Add C to II and then add V.
VII Cool with stirring.
VIII Add G at approx. 35° C.

### Example 3

### Hair Rinse to Counteract Greasy Hair

| | | |
|---|---|---|
| A | Diethanolamine ester quat (according to Ex. 1b) | 1.00% |
| | Cetyl Alcohol | 3.00% |
| | Hostacerin ® DGSB (Clariant) | 1.50% |
| | PEG-4 Polyglyceryl-2-Stearate | |
| B | Water | ad 100% |
| | Sorbitol | 2.00% |
| | Glycerol | 2.00% |
| | D-Panthenol | 0.50% |
| C | Tocopheryl acetate | 0.20% |
| | Extrapone Nettle Special | 2.00% |
| | Aqua, Ethoxydiglycol, Propylene Glycol, | |
| | Butylene Glycol, Nettle (Urtica Dioica) Extract | |
| | Extrapone Melissa Special | 2.00% |
| | Aqua, Ethoxydiglycol, Propylene Glycol, Butylene | |
| | Glycol, Balm Mint (Melissa Officinalis) Extract | |
| | Cosi Silk soluble | 0.30% |
| | Hydrolyzed Silk | |
| | Chitin Liquid | 0.30% |
| | Carboxymethylchitin | |
| | Hydrotriticum WQ | 0.30% |
| | Hydroxypropyltrimonium Hydrolyzed Wheat Protein | |
| | Fragrance | 0.30% |
| | Dye | q.s. |
| | Preservative | q.s. |
| D | Citric acid | q.s. |

### Preparation Method:

### I Melt A at 80° C.

### II Heat B to 80° C.

III Stir II into I.
IV Cool while stirring.

### V Add C to IV at 35° C.

VI Adjust the pH to pH>4.0 with D.

### Example 4

### Pearlizing Hair Rinse

| | | |
|---|---|---|
| A | Genamin ® KSL (Clariant) | 9.00% |
| | PEG-5 Stearyl Ammonium lactate | |
| | Diethanolamine ester quat (according to Ex. 1i) | 1.50% |
| | Hostaphat ® KL 340 D (Clariant) | 1.50% |
| | Trilaureth-4 Phosphate | |
| | Jojoba oil | 1.00% |
| B | Tylose H 100 000 YP2 | 1.50% |
| | Hydroxyethylcellulose | |
| C | Water | ad 100% |
| D | Fragrance | q.s. |
| | Panthenol | 0.50% |
| | Genapol ® PDC (Clariant) | 4.00% |
| | Glycol Distearate/Laureth-4/ | |
| | Cocamidopropyl Betaine/Mica/Titanium Dioxide | |
| E | Citric acid | q.s. |

### Preparation Method:

I Heat components A to 75° C.
II Dissolve B in C and heat to 75° C.
III Add II to I while stirring.
IV Cool to 30° C. while stirring. Successively add components D.
V Adjust the pH with E.

### Example 5

### Hair Rinse Comprising Silicone

| | | |
|---|---|---|
| A | Hostacerin ® DGI (Clariant) | 1.50% |
| | Polyglyceryl-2 Sesquiisostearate | |
| | Cetyl Alcohol | 4.00% |
| B | Diethanolamine ester quat (according to Ex. 1c) | 3.30% |
| | Water | ad 100% |
| C | Fragrance | 0.30% |
| | Preservative | q.s. |
| | Dye | q.s. |
| | SilCare ® Silicone SEA (Clariant) | 1.00% |
| | Trideceth-9 PG-Amodimethicone and Trideceth-12 | |

### Preparation Method:

I Melt A at approx. 70° C.
II Heat B to approx. 70° C.
III Add II to I while stirring and cool while stirring.

### IV Add C to III at 30° C.

### V Adjust to pH 4.0.

### Example 6

### End Fluid, Leave on

| | | |
|---|---|---|
| A | Water | 50.00% |
| B | Tylose ® H 10 000 G4 | 1.00% |
| | Hydroxyethylcellulose | |
| C | Water | ad 100% |
| D | Diethanolamine ester quat (according to Ex. 1d) | 2.50% |
| | Glycerol | 2.00% |
| E | Citric acid | q.s. |

### Preparation Method:

### I Swell B in A,

II Successively dissolve components D in C.

### III Add II to I.

III Adjust the pH with E.

### Example 7

### Haircare Spray

| | | |
|---|---|---|
| A | Diethanolamine ester quat (according to Ex. 1e) | 0.50% |
| | Genamin ® PQ 43 (Clariant) | 2.00% |
| | Polyquaternium-43 | |
| | Genaminox ® CSL (Clariant) | 1.50% |
| | Cocamine Oxide | |
| B | Water | ad 100% |
| C | D-Panthenol | 0.50% |
| | Glycerol | 2.00% |
| | Fragrance | 0.50% |
| | Uvinul ® MS40 | 0.10% |
| | Benzophenone-4 | |
| | Dye | q.s. |
| | Preservative | q.s. |
| D | Emulsogen ® DTC Acid (Clariant) | 2.00% |
| | Trideceth-7-Carboxylic Acid | |
| | SilCare ® Silicone 41M15 (Clariant) | 0.50% |
| | Caprylyl Methicone | |

### Preparation Method:

I Stir components A into B; stir while heating to 60° C. until clarification.
II Cool to room temperature.
III Add C to II and stir until clarification.
IV Homogeneously mix components D and stir into III.

### Example 8

### 2-Phase Conditioner Spray, Leave on

| | | |
|---|---|---|
| A | Genamin ® PDAC (Clariant) | 2.00% |
| | Polyquaternium-6 | |
| | Diethanolamine ester quat (according to Ex. 1c) | 2.00% |
| | Genamin ® CSL (Clariant) | 2.00% |
| | Cocamine Oxide | |
| | D-Panthenol | 0.50% |
| | Glycerol | 2.00% |
| | Fragrance | q.s. |
| | Uvinul MS40 | 0.10% |
| | Benzophenone-4 | |
| | Dye | q.s. |
| | Preservative | q.s. |
| B | Water | ad 100% |
| C | SilCare ® Silicone 41M15 (Clariant) | 15.00% |
| | Caprylyl Methicone | |
| D | NaCl | 0.30% |

### Preparation Method:

I Stir A into B; stir until clarification.

### II Add C to I.

III Add D to II and dissolve with stirring.

### Example 9

### O/W cream

| | | |
|---|---|---|
| A | Diethanolamine ester quat (according to Ex. 1i) | 1.00% |
| | Hostacerin ® DGSB (Clariant) | 5.00% |
| | Polyglyceryl-2 PEG-4 Stearate | |
| | Almond oil | 10.00% |
| | Sunflower oil | 4.00% |
| | Jojoba oil | 2.00% |
| B | Aristoflex ® AVC (Clariant) | 0.60% |
| | Ammonium Acryloyldimethyltaurate/VP Copolymer | |
| C | Tetrasodium EDTA | 0.10% |
| | Ethylenediaminetetraacetic acid, sodium salt | |
| | Citric acid (10% in water) | 0.30% |
| | Glycerol | 3.00% |
| | Water | ad 100% |
| D | Fragrance | 0.40% |
| | Preservative | q.s. |

### Preparation Method:

I Melt A at 70° C., then add B.

### II Heat C to 70° C.

III Stir II into I.
IV Cool while stirring.

### V Add D to IV at 35° C.

### VI Homogenize.

### Example 10

### Pearlizing Conditioning Shampoo

| | | |
|---|---|---|
| A | Diethanolamine ester quat | 2.00% |
| | (according to Ex. 1b) (Clariant) | |
| | Genapol ® PMS (Clariant) | 1.50% |
| | Glycol Distearate | |
| | Genapol ® LRO liquid (Clariant) | 35.00% |
| | Sodium Laureth Sulfate | |
| | Glucamate DOE 120 | 2.50% |
| | PEG-120 Methyl Glucose Dioleate | |
| | Cetyl alcohol | 0.50% |
| | NaCl | 0.50% |
| | Sodium cumenesulfonate | 0.50% |
| | Celquat SC 230 M | 0.30% |
| | Polyquaternium-10 | |
| B | Water | ad 100% |
| C | Genagen ® CAB 818 (Clariant) | 6.00% |
| | Cocamidopropyl Betaine | |
| | Hostapon ® KCG (Clariant) | 8.00% |
| | Sodium cocoylglutamate | |
| D | Fragrance | q.s. |
| | Preservative | q.s. |
| | Fragrance | 0.30% |
| | D-Panthenol | 0.50% |
| | SilCare ® Silicone 41M15 (Clariant) | 0.50% |
| | Caprylyl Methicone | |

### Preparation Method:

I Melt components A at approx. 75° C. and dissolve in B while stirring.
II Cool while stirring.
III Add components C to II.
IV If necessary, adjust the pH.

### V Add D to IV.

Formulation examples 3, 5, 6, 8 and 10 are reworked, except in each case using the diethanolamine ester quats (1e) and (1i) instead of the diethanolamine ester quats (1b), (1c) and (1d).

### Example 11

### Hair Rinse to Counteract Greasy Hair

| | | |
|---|---|---|
| A | Diethanolamine ester quat (according to Example 1f), | 3.00% |
| | 50% active in cetyl alcohol:stearyl alcohol in a | |
| | weight ratio of 70:30 (Ex. 11a), 50:50 (Ex. 11b) | |
| | and 30:70 (Ex. 11c) as pellets | |
| | Cetyl Alcohol | 1.50% |
| | Hostacerin ® DGSB (Clariant) | 1.50% |
| | PEG-4 Polyglyceryl-2-Stearate | |
| B | Water | ad 100% |
| | Sorbitol | 2.00% |
| | Glycerol | 2.00% |
| | D-Panthenol | 0.50% |
| C | Tocopheryl acetate | 0.20% |
| | Extrapone Nettle Special | 2.00% |
| | Aqua, Ethoxydiglycol, Propylene Glycol, | |
| | Butylene Glycol, Nettle (Urtica Dioica) Extract | |
| | Extrapone Melissa Special | 2.00% |
| | Aqua, Ethoxydiglycol, Propylene Glycol, Butylene | |
| | Glycol, Balm Mint (Melissa Officinalis) Extract | |
| | Cosi Silk soluble | 0.30% |
| | Hydrolyzed Silk | |
| | Chitin Liquid | 0.30% |
| | Carboxymethylchitin | |
| | Hydrotriticum WQ | 0.30% |
| | Hydroxypropyltrimonium Hydrolyzed Wheat Protein | |
| | Fragrance | 0.30% |
| | Dye | q.s. |
| | Preservative | q.s. |
| D | Citric acid | q.s. |

### Preparation Method:

### I Melt A at 80° C.

### II Heat B to 80° C.

III Stir II into I.
IV Cool while stirring.

### V Add C to IV at 35° C.

VI Adjust the pH to pH>4.0 with D.

### Example 12

### Hair Rinse Comprising Silicone

| | | |
|---|---|---|
| A | Hostacerin ® DGI (Clariant) | 1.50% |
| | Polyglyceryl-2 Sesquiisostearate | |
| B | Diethanolamine ester quat (according to Example 1h), | 7.50% |
| | 50% active in cetyl alcohol:stearyl alcohol in a | |
| | weight ratio of 70:30 (Ex. 12a), 50:50 (Ex. 12b) | |
| | and 30:70 (Ex. 12c) as pellets | |
| | Water | ad 100% |
| C | Fragrance | 0.30% |
| | Preservative | q.s. |
| | Dye | q.s. |
| | SilCare ® Silicone SEA (Clariant) | 1.00% |
| | Trideceth-9 PG-Amodimethicone and Trideceth-12 | |

### Preparation Method:

I Melt A at approx. 70° C.
II Heat B to approx. 70° C.
III Add II to I while stirring and cool while stirring.

### IV Add C to III at 30° C.

### V Adjust to pH 4.0.

### Example 13

### Pearlizing Conditioning Shampoo

| | | |
|---|---|---|
| A | Diethanolamine ester quat (according to Example 1g), | 3.00% |
| | 50% active in cetyl alcohol:stearyl alcohol in a | |
| | weight ratio of 70:30 (Ex. 13a), 50:50 (Ex. 13b) | |
| | and 30:70 (Ex. 13c) as flakes | |
| | Genapol ® PMS (Clariant) | 1.50% |
| | Glycol Distearate | |
| | Genapol ® LRO liquid (Clariant) | 35.00% |
| | Sodium Laureth Sulfate | |
| | Glucamate DOE 120 | 2.00% |
| | PEG-120 Methyl Glucose Dioleate | |
| | NaCl | 0.50% |
| | Sodium cumenesulfonate | 0.50% |
| | Celquat SC 230 M | 0.30% |
| | Polyquaternium-10 | |
| B | Water | ad 100% |
| C | Genagen ® CAB 818 (Clariant) | 6.00% |
| | Cocamidopropyl Betaine | |
| | Hostapon ® KCG (Clariant) | 8.00% |
| | Sodium cocoylglutamate | |
| D | Fragrance | q.s. |
| | Preservative | q.s. |
| | Fragrance | 0.30% |
| | D-Panthenol | 0.50% |
| | SilCare ® Silicone 41M15 (Clariant) | 0.50% |
| | Caprylyl Methicone | |

### Preparation Method:

I Melt components A at approx. 75° C. and dissolve in B while stirring.
II Cool while stirring.
III Add components C to II.
IV If necessary, adjust the pH.

### V Add D to IV.

Formulation examples 11 to 13 are reworked, except using the diethanolamine ester quats (1e) and (1i) instead of the diethanolamine ester quats (1f), (1g) and (1 h), in each case both as pellets and as flakes, and additionally in each case with 50% active content in cetyl alcohol:stearyl alcohol in a weight ratio of 70:30, 50:50 and 30:70.

### IV. Conditioning Action of Formulations Comprising Compounds of the Formula (1)

### Example 14

### Hair Rinse

| | | |
|---|---|---|
| A | Hostacerin ® T-3 (Clariant) | 1.50% |
| | Ceteareth-3 | |
| | Diethanolamine ester quat (according to Table 2), | 2.00% |
| | 50% active in cetyl alcohol:stearyl alcohol in a | |
| | weight ratio of 50:50 as pellets | |
| | Cetyl Alcohol | 2.00% |
| B | Water | ad 100.00% |
| C | Citric acid (10% in water) | q.s. |

### Preparation Method:

### I Melt A at 80° C.

II Add B to A while stirring and cool while stirring.
III Adjust to pH 4.0 with C.

TABLE 2
Conditioning action of the hair rinse according to Example
14 comprising diethanolamine ester quats 1a-1i

| | 1a | 1b | 1c | 1d | 1e | 1f | 1g | 1h | 1i |
|---|---|---|---|---|---|---|---|---|---|
| Conditioning action - wet | 0 | + | ++ | ++ | + | 0 | + | ++ | ++ |
| Conditioning action - dry | 0 | + | ++ | ++ | ++ | + | ++ | ++ | ++ |

−− = no conditioning action
− = poorer conditioning action compared to the standard
0 = comparable conditioning action to the standard
+ = improved conditioning action compared to the standard
++ = greatly improved conditioning action compared to the standard

The hair rinse according to Example 14, comprising diethanolamine ester quat 1a, constitutes a comparative formulation.

The use of diethanolamine ester quats (according to Table 2), 50% active in cetyl alcohol:stearyl alcohol in a weight ratio of 70:30 and 30:70 as pellets in the hair rinse according to Example 14 leads to the same results with regard to conditioning action as reported in Table 2.

1. A composition comprising one or more compounds of the formula (1) 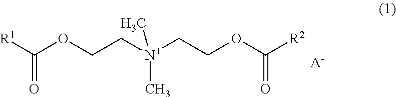
in which
R¹CO and R²CO are each independently linear or branched saturated acyl groups having 18 to 24 carbon atoms and
A⁻ is a counterion
and the total amount of C₁₈₋₂₃-alkyl-COO— groups, based on all R¹COO— and R²COO— groups, is 40.0% by weight or more.

2. The composition as claimed in claim 1, wherein R¹ and R² are each independently linear alkyl groups having 17 to 23 carbon atoms.

3. The composition as claimed in claim 1 or 2, wherein a plurality of compounds of the formula (1) are present therein, and the amount of C₁₇-alkyl-COO— groups of the compounds of the formula (1), based on all R¹COO— and R²COO— groups, is from 1.0 to 45.0% by weight, the amount of C₁₉-alkyl-COO— groups of the compounds of the formula (1), based on all R¹COO— and R²COO— groups, is from 1.0 to 15.0% by weight, and the amount of C₂₁-alkyl-COO— groups of the compounds of the formula (1), based on all R¹COO— and R²COO— groups, is from 42.0 to 94.0% by weight.

4. The composition as claimed in one or more of claims 1 to 3, wherein a plurality of compounds of the formula (1) are present therein, and the amount of C₁₇-alkyl-COO— groups of the compounds of the formula (1), based on all R¹COO— and R²COO— groups, is from 41.0 to 45.0% by weight, the amount of C₁₉-alkyl-COO— groups of the compounds of the formula (1), based on all R¹COO— and R²COO— groups, is from 9.0 to 13.0% by weight, and the amount of C₂₁-alkyl-COO— groups of the compounds of the formula (1), based on all R¹COO— and R²COO— groups, is from 42.0 to 46.0% by weight.

5. The composition as claimed in one or more of claims 1 to 3, wherein a plurality of compounds of the formula (1) are present therein, and the amount of C₁₇-alkyl-COO— groups of the compounds of the formula (1), based on all R¹COO— and R²COO— groups, is from 1.0 to 6.0% by weight, the amount of C₁₉-alkyl-COO— groups of the compounds of the formula (1), based on all R¹COO— and R²COO— groups, is from 12.0 to 15.0% by weight, and the amount of C₂₁-alkyl-COO— groups of the compounds of the formula (1), based on all R¹COO— and R²COO— groups, is from 79.0 to 84.0% by weight.

6. The composition as claimed in one or more of claims 1 to 3, wherein a plurality of compounds of the formula (1) are present therein, and the amount of C₁₇-alkyl-COO— groups of the compounds of the formula (1), based on all R¹COO— and R²COO— groups, is from 1.0 to 6.0% by weight, the amount of C₁₉-alkyl-COO— groups of the compounds of the formula (1), based on all R¹COO— and R²COO— groups, is from 1.0 to 6.0% by weight, and the amount of C₂₁-alkyl-COO— groups of the compounds of the formula (1), based on all R¹COO— and R²COO— groups, is from 90.0 to 94.0% by weight.

7. The composition as claimed in one or more of claims 1 to 6, which comprises one or more compounds of the formula (2) 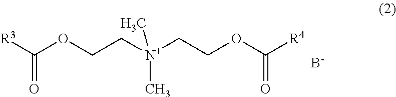
in which
R³CO and R⁴CO are each independently linear or branched saturated acyl groups having 12 to 24 carbon atoms, preferably having 18 to 24 carbon atoms, or linear or branched, mono- or polyunsaturated acyl groups having 12 to 24 carbon atoms, preferably having 18 to 24 carbon atoms, where at least one of the R³CO and R⁴CO groups must be a mono- or polyunsaturated acyl group, and
B⁻ is a counterion and
the amount of the compounds of the formula (2), based on the total amount of the compounds of the formulae (1) and (2), is less than 20.0% by weight.

8. The composition as claimed in claim 7, wherein the amount of the compounds of the formula (2), based on the total amount of the compounds of the formulae (1) and (2), is less than 10.0% by weight.

9. The composition as claimed in one or more of claims 1 to 8, wherein no compound of the formula (2) is present.

10. The composition as claimed in one or more of claims 1 to 9, wherein the counterions A⁻ or A⁻ and B⁻ are each independently selected from chloride, bromide, methosulfate MeSO₄⁻, tosylate, phosphate, sulfate, hydrogensulfate, lactate and citrate.

11. The composition as claimed in one or more of claims 1 to 10, wherein the counterions A⁻ or A⁻ and B⁻ are each independently selected from chloride and methosulfate MeSO₄⁻.

12. The composition as claimed in one or more of claims 1 to 11, which comprises, based on the overall composition, from 0.1 to 10.0% by weight of the one or more compounds of the formula (1).

13. The composition as claimed in one or more of claims 1 to 12, which comprises, based on the overall composition, from 1.0 to 5.0% by weight of the one or more compounds of the formula (1).

14. The composition as claimed in one or more of claims 1 to 13, which comprises one or more unbranched or branched monoalcohols with an alkyl group having 8 to 22 carbon atoms, and preferably, based on the overall composition, from 0.1 to 70.0% by weight of one or more of these substances.

15. The composition as claimed in one or more of claims 1 to 14, which comprises one or more monoalcohols with an alkyl group having 1 to 7 carbon atoms, and preferably, based on the overall composition, from 0.1 to 70.0% by weight of one or more of these substances.

16. The composition as claimed in one or more of claims 1 to 15, which comprises one or more polyols having 3 to 12 carbon atoms, and preferably, based on the overall composition, from 0.1 to 70.0% by weight of one or more of these substances.

17. The composition as claimed in one or more of claims 1 to 16, which comprises diethanol/methylamine, and preferably, based on the overall composition, from 10 ppm to 1.0% by weight of diethanol/methylamine.

18. The composition as claimed in one or more of claims 1 to 17, which comprises one or more fatty acids of the formulae R^{1a}COOH and R^{2a}COOH, in which R^{1a}CO and R^{2a}CO are each independently linear or branched acyl groups having 18 to 24 carbon atoms, preferably linear or branched saturated acyl groups having 18 to 24 carbon atoms, and preferably, based on the overall composition, from 10 ppm to 1.0% by weight of one or more of these substances.

19. The composition as claimed in one or more of claims 1 to 18, which comprises one or more compounds of the formula (3) 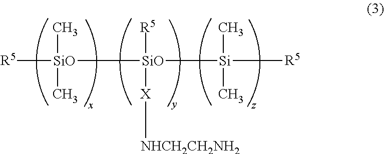
in which
R⁵ is —OH or —CH₃,
X is a linear or branched alkylene group having 1 to 6 carbon atoms,
x, y and z are each independently numbers from 1 to 5500, preferably from 50 to 500,
and preferably, based on the overall composition, from 0.1 to 5.0% by weight of one or more compounds of the formula (3).

20. The composition as claimed in one or more of claims 1 to 19, which comprises one or more compounds of the formula (4) 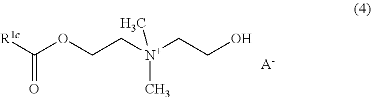
in which
R^{1c}CO is a linear or branched, preferably linear, additionally preferably saturated, acyl group having 18 to 24 carbon atoms, preferably 18 to 22 carbon atoms, and
A⁻ is a counterion,
and preferably, based on the overall composition, from 0.1 to 5.0% by weight of one or more compounds of the formula (4).

21. The composition as claimed in claim 20, wherein the counterion A⁻ is selected from chloride, bromide, methosulfate MeSO₄⁻, tosylate, phosphate, sulfate, hydrogensulfate, lactate and citrate, and is preferably selected from chloride and methosulfate MeSO₄⁻.

22. The composition as claimed in one or more of claims 1 to 21, which is in the form of a dispersion.

23. The composition as claimed in one or more of claims 1 to 22, which comprises one or more nonionic emulsifiers, and preferably, based on the overall composition, from 0.1 to 5.0% by weight of one or more nonionic emulsifiers.

24. The composition as claimed in one or more of claims 1 to 23, which is a cosmetic, dermatological or pharmaceutical composition.

25. The composition as claimed in one or more of claims 1 to 24, which is an oil-in-water emulsion.

26. The composition as claimed in one or more of claims 1 to 25, which has a pH in the range from 2 to 12, preferably in the range from 3 to 9.

27. The composition as claimed in one or more of claims 1-11, 17, 18, 20 and 21, which is in the form of pellets or flakes and preferably contains from 45.0 to 70.0% by weight of one or more compounds of the formula (1) and from 30.0 to 55.0% by weight of one or more monoalcohols with an unbranched or branched alkyl group having 8 to 22 carbon atoms.

28. The use of a composition as claimed in one or more of claims 1 to 26 for treatment or care of the skin.

29. The use of a composition as claimed in one or more of claims 1 to 26 for treatment or care of hair.
